(19) United States
(12) Patent Application Publication
(10) Pub. No.: US 2012/0082732 A1
(43) Pub. Date: Apr. 5, 2012

(54) MEDICINE FOR TREATMENT OF A CARCINOMA

(76) Inventors: JENS FEHRE, Hausen (DE); Ralf Nanke, Neunkirchen am Brand (DE); Martin Stetter, Muenchen (DE)

(21) Appl. No.: 13/313,664

(22) Filed: Dec. 7, 2011

Related U.S. Application Data
(62) Division of application No. 12/203,204, filed on Sep. 3, 2008.

(30) Foreign Application Priority Data
Sep. 3, 2007 (DE) 10 2007 041 834.7

(51) Int. Cl. A61K 38/17; A61K 9/14; A61K 31/7088; A61P 37/04; A61K 35/76; A61K 39/395; A61P 35/00

(52) U.S. Cl. 424/499; 424/193.1; 424/133.1; 424/196.11; 424/178.1

(57) ABSTRACT

In a method for treating a carcinoma in a patient, a medicine is administered via the blood stream of the patient that appears, to the patient's immune system, that tissue of the carcinoma is an inflammation source. The medicine employs two active components that are coupled to each other in a form allowing administration of the two active components to the carcinoma via the blood stream of the patient. A first of the active components is at least one coupling molecule that specifically tethers to a target molecule formed by cancer tissue of the carcinoma. A second of the active ingredients is at least one signal molecule typical to inflammation, or at least one originating molecule encoding such a signal molecule, that induces the immune system of the body to attack the cancer cells.

### RELATED APPLICATION

The present application is a divisional application Ser. No. 12/203,204, filed Sep. 3, 2008 (now abandoned), from which the present application claims the benefit of the earlier filing date under 35 U.S.C. §120.

### BACKGROUND OF THE INVENTION

1. Field of the Invention

The present invention concerns a medicine for treatment of a carcinoma, in particular prostate cancer.

2. Description of the Prior Art

In the event of inflammation, foreign cells or foreign bodies that have penetrated into the body tissue are attacked and destroyed (primarily by leukocytes) or rendered harmless. In contrast to this, carcinomas have the property that they are not attacked by the immune system of the body, such that in many cases they are only noticed when a successful treatment (for instance against metastasis formation) is barely possible.

### SUMMARY OF THE INVENTION

An object of the present invention is to provide a medicine that induces the immune system of the body to attack cancer cells.

This object is achieved by a medicine according to the invention that can be supplied via the circulatory system, and thus can be administered intravenously, orally or rectally (for example), and that contains a first active component and a second active component that are coupled with one another. The coupling can ensue, for example, by a direct chemical bond or an indirect association by, for instance, the active components being bound immobilized to a carrier, for instance being enclosed by the carrier or being bound chemically or in another manner to the carrier. The first active component is formed from at least one molecule (designated in the following as a coupling molecule) that specifically binds to the target molecule formed by the cancer tissue. The second active component is formed from at least one signal molecule typical to inflammation, or from at least one originating molecule that can be transformed into such a signal molecule. Due to the coupling molecules of the first active component, the medicine selectively accumulates in the vascular endothelium of the cancer tissue. Due to the signal molecules of the second active component that are typical to inflammation, the cancer tissue presents itself as an inflammation source that is attached by the immune system of the body. As explained in detail below, this leads to a fixation of leukocytes to the signal molecules. A relatively high probability thereby exists that at least a portion of the leukocytes migrate across the vessel wall into the cancer tissue, so the signal or reaction cascades that are specific to inflammation are triggered and at least a portion of the cancer cells are attacked and killed. The medicine according to the invention thus causes a targeted inducement of the self-healing powers of the body.

### BRIEF DESCRIPTION OF THE DRAWINGS

FIG. 1 illustrates the selective accumulation of a medicine in the vascular endothelium of a cancer tissue.

FIGS. 2 and 3 illustrate the manner of operation of a medicine in accordance with the invention having second active component that contains a plasmid as an originating molecule.

### DESCRIPTION OF THE PREFERRED EMBODIMENTS

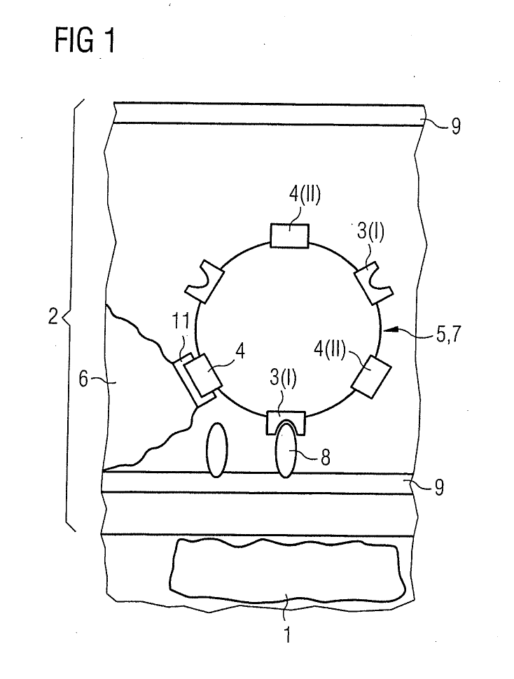

A cancer tissue 1 (for example a prostate carcinoma) and blood vessel 2 permeating this or directly adjacent to this are respectively indicated in the images. In the exemplary embodiment according to FIG. 1, a medicine that contains a first active component I that is formed from multiple coupling molecules 3 and a second active component II that is formed from multiple, inflammation-specific signal molecules 4 is supplied to the cancer tissue 1 via the circulatory system. A carrier 5 holding a coupling molecule and signal molecule together is present as an additional component. The design of the carrier 5 is arbitrary in principle. For example, the carrier 6 can be a protein to which at least one coupling molecule 3 and at least one signal module 4 are respectively bound, but microbubbles 7 are advantageously used as the carrier 5. These are microscopically small hollow spheres having an envelope formed from a lipid double membrane, for example. The mutual binding of coupling molecules and signal molecules 4 is achieved by the cited molecules being present inside the microbubbles 7, or being incorporated into its envelope and/or being bound to its outside. In the case shown in FIG. 1, the coupling molecules 3 and signal molecules 4 are connected with the outside of the microbubble 7.

The coupling molecules 3 are such molecules or molecular structures that bind to target molecules 8 that form in the endothelium 9 of a blood vessel 2. In the preliminary stage of the carcinoma, this has not yet developed blood vessels 2. In this case the target molecules 8 are in the endothelium of blood vessels 2 directly adjacent to the cancer tissue. The CEACAM-1 molecule (carcinoembryonic antigen-related cell adhesion molecule) is formed in the preliminary stage of high-grade intraepithelial neoplasy (for instance of the prostate; hgPIN), for example. Coupling molecules 3 specifically binding to this are, for example, CEACAM-1 antibodies. Carcinomas that have progressed further induce angiogenesis, the formation of blood vessels 2. The growth factors accompanying angiogenesis—such as, for example, VEGF (vasco endothelial growth factor) or Alpha(V)-beta(3)-integrin—thereby serve as target molecules 8 with which corresponding coupling molecules 3 interact as binding partners, for example Alpha(V)-beta(3)-integrin ligands or respective corresponding antibodies in the cited case. So that carcinomas in the preliminary stage and those that are already located in the angiogenesis stage can be detected and treated, the medicine can contain the respective, specifically binding coupling molecules 3 in combination.

Furthermore, molecules known as aptamers are suitable for use as the coupling molecules 3 in the cited cases. These are short, stable and specifically binding RNA chains. Anticalins also are suitable as the coupling molecules 3. These are individual polypeptide chains with approximately 180 amino acids that have specific binding properties similar to antibodies, but are easier to produce.

As already mentioned, the second active component of a medicine according to the invention has the task of pretending to the body's own defense system that cancer tissue is an inflammation source. Corresponding to this goal, the second active component II is formed by at least one signal molecule 4 typical to an inflammation. An inflammation process runs over multiple different stages with which separate signal molecules are respectively associated. However, not all of these signal molecules are suitable for the provided purpose. The attraction and the accumulation of leukocytes in the region of an inflammation source is sub-divided into the phases of tethering, rolling, possible activation, fixed adhesion and transendothelial migration. A separate pair made up of adhesion molecule and leukocyte-persistent ligands binding to said adhesion molecule is responsible for each phase. The initial tethering and the rolling of the leukocytes occurs in that CLA molecules (cutaneous lymphocyte-associated antigen molecules) temporarily bind to E-selectin in the endothelial membrane in the leukocyte membrane. The leukocytes are thereby decelerated from blood stream by a factor of approximately 100. In the next step, leukocytes are activated via chemokines and are thereby put in the position of tethering to VCAM-1 molecules (vascular cytokine activated adhesion molecules) with the use of leukocyte-persistent integrins. An additional adhesion initially ensues. As a result, LFA-1 (leukocyte function antigen) of the leukocytes can tether to ICAM-1 (intercellular cytokine-activated adhesion molecule) in the endothelium, whereby the leukocyte is immobilized. Signal cascades are triggered both in the endothelium and in the leukocytes due to the immobilization, which signal cascades finally cause the migration of the leukocytes into the affected tissue, followed by an inflammation reaction. The binding between VCAM-1 and leukocyte-persistent integrins (for example the alpha-(4)-beta (1) integrin of the skin) followed by the binding between ICAM-1 and leukocyte-persistent LFA-1 represent key processes in many different tissue types. These are therefore particularly suitable to achieve the goal according to the invention, namely to pretend to the immune system that cancer tissue is an inflammation source. Cytokine-activated adhesion molecules (CAM), advantageously VCAM-1 and ICAM-1, are therefore used as inflammation-specific signal molecules 4. In the exemplary embodiment according to FIG. 1, these are connected with the outside of the microbubble 7 (or with a carrier 5 designed in another manner). As mentioned above, the leukocytes are activated by chemokines and thereby put in the position to bind to VCA-1 molecules with the aid of leukocyte-persistent integrins. To increase the effectiveness of the proposed medicine, it is therefore appropriate to attach at least one chemokine of the cited type as a third active component. With regard to the effectiveness (thus the certain triggering of the inflammation-specific reactions), it is advantageous if the medicine contains both VCAM-1 and ICAM-1 as signal molecules 4.

In the exemplary embodiment shown in FIG. 1, a leukocyte 6 with a ligand 11 tethers to the signal molecule 4 of the carrier 5, which does not entirely correspond to the conditions in actual inflammation processes since here the signal molecules are localized in a vascular endothelium 9. In order to get closer to the actual ratios, and therefore to increase the probability of the migration of a leukocyte 6 into endothelial cells and the triggering of inflammation-specific signal cascades, in a variant of the medicine it is provided to use microbubbles 7 as a carrier 5, which microbubbles 7 can be destroyed by charging with ultrasound (for example via a rectal probe). Given the destruction (bursting) of the microbubbles 7, the signal molecules in or on them are released. Moreover, the effect known as sonoporation (the endothelium is temporarily destabilized) is connected with the ultrasonic charging. Pores into which the signal molecules partially penetrate thereby form in the cell membranes, such that after the ultrasonic charging they are anchored in the endothelium as with normal inflammation sources.

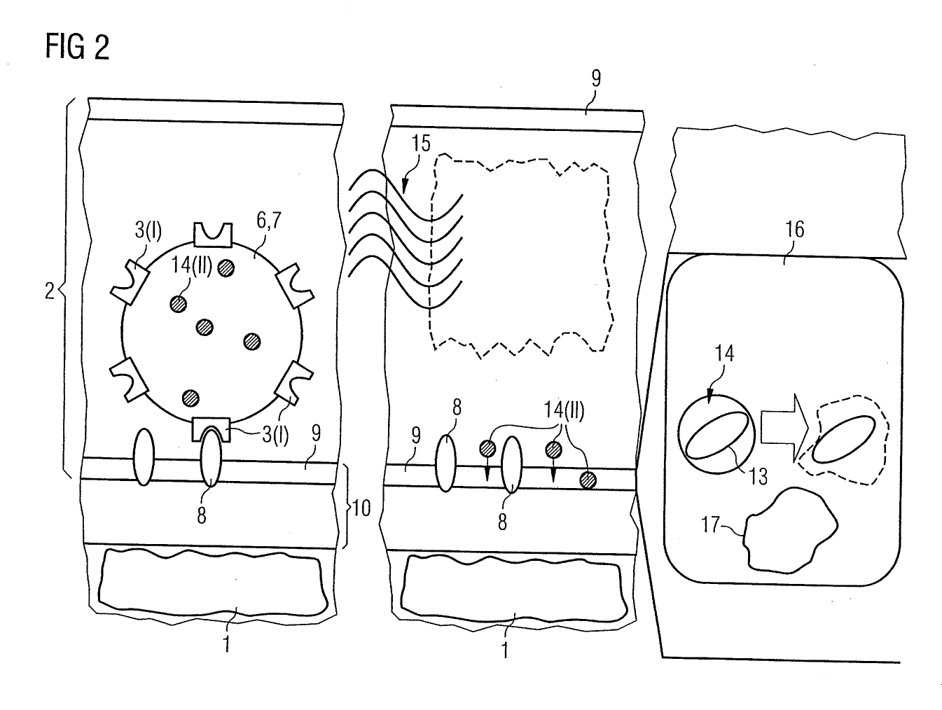

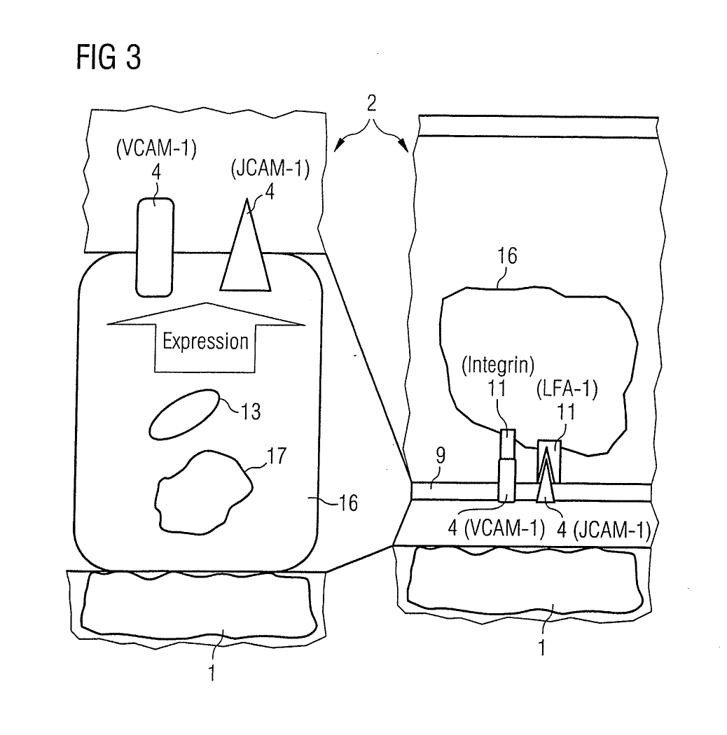

In a preferred embodiment of the medicine, the signal molecules 4 are transported to the target tissue not as such but rather in the form of an originating molecule (in particular a plasmid) encoding them. The actual target molecule is only formed at the target location and in fact within an endothelial cell. The introduction of the originating molecule or, respectively, the plasmid into the endothelial cells ensues by transient transfection, which is shown in FIGS. 2 and 3. A microbubble 7 acting as a carrier 5 carries coupling molecules 3 (for example CEACAM-1 ligands) on its surface. These couple to target molecules 8 (for example CEACAM-1 molecules), whereby the microbubble is immobilized on the endothelium 9. The second active component II—which contains a plasmid 13 encoding CAM (cell adhesion molecule), for example—is located within the microbubble 7. A viral vector, i.e. a virus 14 that is harmless to humans, serves as a transport vehicle to transfer the plasmid into an endothelial cell. The envelope of the microbubble 7 is destroyed by the action of ultrasonic waves 15 and the viruses 14 thus are released. Possibly supported by the effect of the sonoporation, the viruses penetrate into the endothelium 9 or, respectively, into endothelial cells 16. The plasmid 13 is released there. As given viral illnesses, the host cell (more precisely the cell nucleus 17) reads the plasmid DNA and expresses the gene coded in this. The corresponding proteins—ICAM and/or VCAM molecules in the present case—are thus formed. In contrast to an external supply of CAM molecules or other inflammation-specific signal molecules 4, a significantly greater number of signal molecules 4 which are incorporated into the vascular endothelium 9 are available due to the continuous reproduction as a result of gene expression. The reaction triggered by the immune system, beginning with the immobilization of leukocytes in the cancer tissue, therefore occurs to a correspondingly greater degree. For example, a plasmid 13 which expresses both VCAM-1 and CAM-1 as inflammation-typical signal molecules infiltrates into the endothelial cells 16. A leukocyte 17 with an integrin or LFA-1 ligand 11 couples to the cited signal molecules 4. In addition to inflammation-typical signal molecules 4, the genetic information for other proteins (for example for chemokines) which activate the leukocytes 17 and thereby are placed in the position of tethering to VCAM-1 molecules with the use of leukocyte-persistent integrins can also be present in the plasmid 13. The destruction (mentioned above) of the microbubbles 7 with the use of ultrasound is not absolutely necessary. Microbubbles 7 can also be used that dissolve in blood serum. In this case, an envelope that retards dissolving can be appropriate that, given an oral, rectal or intravenous administration, endures at least until immobilization of the microbubbles 7 in the blood vessels 2 of the cancer tissue 1, or in blood vessels adjacent thereto.

Although modifications and changes may be suggested by those skilled in the art, it is the intention of the inventors to embody within the patent warranted hereon all changes and modifications as reasonably and properly come within the scope of their contribution to the art.

I claim as my invention:

1. A method for treatment of a carcinoma in a patient, comprising:
administering a medicine via the blood stream of the patient that appears to the immune system of the body of the patient that tissue of the carcinoma is an inflammation source, said medicine comprising two active components coupled to each other in a form allowing administration of the two active components to the carcinoma via the blood stream of the patient;
employing, as said first of said active components, at least one coupling molecule that specifically tethers to a target molecule formed by cancer tissue of the carcinoma; and
employing, as a second of said active components, at least one signal molecule typical to inflammation, or at least one originating molecule encoding at least one signal molecule typical to inflammation, that induces the immune system of the body of the patient to attack cancer cells in said cancer tissue.

2. A medicine according to claim 1 wherein the first of the active components comprises a coupling molecule that tethers to a target molecule formed in a preliminary stage of the carcinoma.

3. A method according to claim 2 wherein said coupling molecule is a coupling molecule that tethers to a target molecule formed in high-grade intraepithelial neoplasy.

4. A method according to claim 2 wherein said coupling molecule is a coupling molecule that tethers to CEACAM-1 as the target molecule.

5. A method according to claim 4 wherein said coupling molecule is a CEACAM-1 antibody.

6. A method according to claim 1 wherein the first of the active components comprises a coupling molecule that tethers to a target molecule formed in an angiogenesis stage of the carcinoma.

7. A method according to claim 6 wherein said coupling molecule is a coupling molecule that tethers to the growth factor VEGF.

8. A method according to claim 7 wherein said coupling molecule is a VEGF ligand or a VEGF antibody.

9. A method according to claim 3 wherein said coupling molecule is a coupling molecule that binds to integrin.

10. A method according to claim 9 wherein said coupling molecule binds to alpha(V)beta(3) integrin.

11. A method according to claim 10 wherein said coupling molecule is a VEGF ligand or a VEGF antibody.

12. A method according to claim 1 wherein the first of the active components comprises a coupling molecule that tethers to a target molecule formed in a preliminary stage of the carcinoma and a coupling molecule that tethers to a target molecule formed in an angiogenesis stage of the carcinoma.

13. A method according to claim 12 wherein said coupling molecule is a coupling molecule that tethers to a target molecule formed in high-grade intraepithelial neoplasy.

14. A method according to claim 12 wherein said coupling molecule is a coupling molecule that tethers to CEACAM-1 as the target molecule.

15. A method according to claim 14 wherein said coupling molecule is a CEACAM-1 antibody.

16. A method according to claim 6 wherein said coupling molecule is a coupling molecule that tethers to the growth factor VEGF.

17. A method according to claim 16 wherein said coupling molecule is a VEGF ligand or a VEGF antibody.

18. A method according to claim 6 wherein said coupling molecule is a coupling molecule that binds to integrin.

19. A method according to claim 18 wherein said coupling molecule binds to alpha(V)beta(3) integrin.

20. A method according to claim 18 wherein said coupling molecule is a VEGF ligand or a VEGF antibody.

21. A method according to claim 1 comprising at least one coupling molecule selected from the group consisting of anticalins and aptamers.

22. A method according to claim 1 wherein the second of the active components comprises a cytokine-activated adhesion molecule.

23. A method according to claim 22 wherein said second active components is VCAM-1 and/or ICAM-1.

24. A method according to claim 1 comprising including a third active component in said medicine administered to the blood stream of the patient, comprising at least one substance that activates the adhesion of leukocytes to inflammation-specific signal molecules.

25. A method according to claim 24 comprising including at least one chemokine as said third active component.

26. A method according to claim 1 wherein the second of the active components contains, as an originating molecule, a plasmid comprising the genetic code for at least one inflammation-specific signal molecule.

27. A method according to claim 26 comprising including a third active component in said medicine administered to the blood stream of the patient that contains at least one chemokine.

28. A method according to claim 26 comprising using, as said plasmid, a plasmid comprising the genetic code for at least one chemokine.

29. A method according to claim 26 comprising using, as said plasmid, a virus containing a plasmid.

30. A method according to claim 1, comprising microbubbles that carry the active components, said microbubbles being destroyable by ultrasound.
